(19) United States
(12) Patent Application Publication
(10) Pub. No.: US 2021/0022636 A1
(43) Pub. Date: Jan. 28, 2021

(54) BIO-SIGNAL DETECTING HEADBAND

(71) Applicant: MINDSALL, INC., Newark, CA (US)

(72) Inventor: Limin ZHU, Fremont, CA (US)

(21) Appl. No.: 16/520,193

(22) Filed: Jul. 23, 2019

(51) Int. Cl. A61B 5/0478; A61B 5/00; A61B 5/04; A61B 5/0488

(52) U.S. Cl. CPC A61B 5/0478; A61B 5/6803; A61B 5/02438; A61B 5/0488; A61B 5/04012

(57) ABSTRACT

A sensor device measures bio-signals including one or more of electroencephalogram (EEG), heartbeat, electromyography (EMG), body temperature, body location, time, movement and velocity. The sensor device can include a circular fabric based headband with electrodes (conductive fabric sensing material) embedded therein, along with electronical circuitry and electrical wires. Electrodes can be positioned to make electrical contact with the scalp of a subject. For example, electroencephalography (EEG) measures the voltage changes resulting from ionic current with the neurons of the brain over a time period. By using multiple electrodes in contact with the scalp of the subject, neural oscillations can be detected in EEG measurements.

### BACKGROUND OF THE INVENTION

### 1. Field of the Invention

Embodiments of the present invention relates generally to bioelectrical signal and other biological signal detecting sensor devices. More particularly, the invention relates to a headband for measuring bio-signals including one or more of electroencephalogram (EEG), electrocardiogram (ECG), heartbeat, electromyography (EMG), body temperature, body location, time, movement and velocity.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

It is well known that brain function can be monitored by using electrophysiological monitoring and recording electrical activity. Electrodes are positioned to make electrical contact with the scalp of a subject. For example, electroencephalography (EEG) measures the voltage changes resulting from ionic current with the neurons of the brain over a time period. By using multiple electrodes in contact with the scalp of the subject, neural oscillations can be detected in EEG measurements.

Measurement of electric signals from the brain can be used for a number of purposes, such as those described below.

Electrical signals from the brain can be used as a controller for device. As long as the measuring system can differentiate different signals, and production of such signals is in the control of a wearer, the user can use such signals to control the external device. There has been some use of brain waves for control of toys, and some investigation of use of brain waves by the disabled, who may be unable to use hands to control a device.

Electrical signals can be used to diagnose, through abnormal readings, certain medical conditions, such as sleep disorders. One advantage is the ability to measure fine resolution temporal events, which cannot be imaged by other imaging system.

Electrical signals can also be used in cognitive studies to provide greater information about brain function.

There are other bio-signals or physical indicates of time and movements while those bio-signals to be collected, such as electrocardiogram (ECG), heartbeat, electromyography (EMG), body temperature, body location, time, movement and velocity, people used added-on various wearable devices to collect those signals for further analysis, but there is no one general device to collect all those signals simultaneously. As of today, there are two major product types in wearable market, the first one is wristband, and the second is smart watch. Wristband detects activities such as step, heart rate, calorie burning and sleeps status. Smart watch is the extension of mobile phone which inherits market expansion difficulty due to function similarity. To combine all those bio-signals detected simultaneously, it offers extended capability of the correlation analysis among those signals.

For today's solution for brainwave EEG detection and analysis, there are fundamental issues associated which result in barrier for mass market acceptance.

An EEG detection device goes through majorly two stages of development. The first one is as purely medical equipment used in hospital or lab environment with special setup conditions, usually the sensors used for electrical EEG signal are called “wet sensors” with conductive gel to be injected before using. Developers tried to move this brainwave detection technology from medical use conditions to normal consumer environments with dry sensor technology.

However, the form factor of detection device design has no ability to withstand prolonged wear due to the hard plastic or rubber materials, sometimes requiring a painful ear clip. Also, for current existing “dry sensor” solutions, it requires direct skin contact in order to pick up electronic signals, in case there is need for EEG detection in a hairy area, direct sensor-scalp contact is needed through conductive gel injection, or the user must shave the hairs off or use a special comb-shaped sensor to allow direct scalp contact.

In term of current hardware solutions, (1) current available headset or headband is not suitable for active sports use environment, (2) the major building material is plastic, which is bulky and inflexible, (3) the ear clips used in conventional solutions are not comfortable for long time wearing, even resulting pinch pain.

### SUMMARY OF THE INVENTION

It is very easy for people to wear a regular sports fabric headband for long time, without uncomfortable feeling. However, to integrate electrical wires, electrical circuitry PCB module and adjust “soft” conductive fabric for tiny bio-signals (10⁻⁶ V to 10⁻³ V range) detection, it's very difficult. With the highly integrated bio-signal detection modules plus wireless communication and rechargeable battery solutions, embodiments of the present invention provide true garment sports headband which is the world's first prolonged wearable full fabric bio-signal headband. Besides only EEG signal, it can also detect other bio-signals as described above. It keeps the same form factor of regular sports headband but with all needed bio-signal detection functions seamlessly integrated.

Besides EEG signal detection, developers can use add-on wearable devices, such as wrist bands, to detect other bio-signals such as ECG, and body location, however, there is no bio-signal headband solution to have comprehensive multiple bio-signals detection capability, and the correlation between those simultaneously detected bio-signal data can provide more meaningful health information.

Embodiments of the present invention provide a headband comprising a fabric band configured to fit about a user's head; an electrode contact formed on an inside portion of the headband, the electrode contact configured to receive an electroencephalogram (EEG) signal from the user; and a circuit for receiving the EEG signal from the electrode contact.

Embodiments of the present invention further provide a headband comprising a fabric band configured to fit about a user's head; an electrode contact formed on an inside portion of the headband, the electrode contact configured to receive an electroencephalogram (EEG) signal from the user; an auxiliary electrode contact formed on another inside portion of the headband, the auxiliary electrode contact configured to receive a second EEG signal from the user; and a circuit for receiving the EEG signal from the electrode contact and the second EEG signal from the auxiliary electrode contact, the circuit correlating the EEG signal and the second EEG signal into a dual EEG.

Embodiments of the present invention also provide a headband comprising a fabric band configured to fit about a user's head; an electrode contact formed on an inside portion of the headband, the electrode contact configured to receive an electroencephalogram (EEG) signal from the user; an auxiliary electrode contact formed on another inside portion of the headband, the auxiliary electrode contact configured to receive a second electronic signal from the user; one or more embedded sensors, embedded in at least one of the electrode contact and the auxiliary electrode contact; and a circuit for receiving the EEG signal from the electrode contact, the second signal from the auxiliary electrode contact and a signal from the one or more embedded sensors.

Embodiments of the present invention also provide a fabric hat comprising a free touch bio-signal detection module for hairy areas. In contrast to wet and dry contact sensors, the free touch electrostatic sensor does not require an ohmic connection to the body. For body sensor applications, this offers numerous advantages since free touch sensors require zero preparation, are completely insensitive to skin conditions and can be embedded within comfortable layers of fabric.

Besides bio-signal detection for the human body, embodiments of the present invention can also be used for animal bio-signal detection, including animal brainwave electronic signal, and heartbeat electronic signal, and breath electronic signal. Further technology developments can be used for diagnosis and tracking disease conditions and behavior of livestock or pets.

These and other features, aspects and advantages of the present invention will become better understood with reference to the following drawings, description and claims.

### BRIEF DESCRIPTION OF THE DRAWINGS

Some embodiments of the present invention are illustrated as an example and are not limited by the figures of the accompanying drawings, in which like references may indicate similar elements.

FIG. 1A illustrates a front view of a user's head wearing an electronic headband according to an exemplary embodiment of the present invention;

FIG. 1B illustrates a left side view of the user's head wearing the electronic headband of FIG. 1;

FIG. 1C illustrates a back and left side view of the user's head wearing the electronic headband of FIG. 1;

FIG. 2 illustrates a top perspective view of the headband of FIG. 1A, showing the electrical circuitry box and connection wires and conductive sensing fabrics, noting one or more of such elements may not be visible on the exterior of the headband;

FIG. 3A illustrates a headband prototype that a user is wearing;

FIG. 3B illustrates a back perspective view of the headband of FIG. 1A;

FIG. 3C illustrates a top view of the headband of FIG. 1A;

FIG. 4A illustrates a detailed back view of the headband of FIG. 1A, showing the electrically conductive material that is configured for placement against the user's head;

FIG. 4B illustrates a detailed side view of the headband of FIG. 1A, showing the electrically conductive material that is configured for placement against the user's ear region;

FIG. 5A illustrates a back view of the headband of FIG. 1A, showing the protective box electrically disconnected from the headband and partially removed from a bracketing feature on the headband;

FIG. 5B illustrates a detailed perspective view of the bracketing feature of the headband of FIG. 1A, showing the protective box fully removed therefrom;

FIG. 6 illustrates a printed circuit board design usable in the headband of FIG. 1A;

FIG. 7 illustrates bio-signal connection and manufacture process for the headband of FIG. 1A;

FIG. 8 illustrates an exemplary electronic shield wire design detail, usable in the headband of FIG. 1A;

FIG. 9 illustrates an exemplary shielded wire usable in the headband of FIG. 1A;

FIG. 10 illustrates an exemplary embodiment of a pin/conductive fabric wearable solution usable in the headband of FIG. 1A;

FIG. 11 illustrates a multiple brainwave headbands operation solution, according to an exemplary embodiment of the present invention;

FIGS. 12A and 12B illustrate direct communication operation of the headband of FIG. 1A;

FIG. 13A and FIG. 13B illustrate a free touch bio-signal module with electrodes and signal process PCBA module inside protective box;

FIG. 14 illustrates a free touch bio-signal module for placement in a hairy area to detect bio-signals without direct skin contact;

FIG. 15 and FIG. 16 illustrate a bio-signal detection PCB module that can be attached on a cow's head; and

FIG. 17 and FIG. 18 illustrate bio-signal detection for a dog low abdomen area, including an output signal therefrom.

Unless otherwise indicated illustrations in the figures are not necessarily drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary, a variety of optional components are described to illustrate the wide variety of possible embodiments of the present invention.

A “computer” or “computing device” may refer to one or more apparatus and/or one or more systems that are capable of accepting a structured input, processing the structured input according to prescribed rules, and producing results of the processing as output. Examples of a computer or computing device may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

“Software” or “application” may refer to prescribed rules to operate a computer. Examples of software or applications may include: code segments in one or more computer-readable languages; graphical and or/textual instructions; applets; pre-compiled code; interpreted code; compiled code; and computer programs.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hypertext Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Smalltalk, Python, Perl, UNIX Shell, Visual Basic or Visual Basic Script, Virtual Reality Markup Language (VRML), ColdFusion™ or other compilers, assemblers, interpreters or other computer languages or platforms.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). The program code may also be distributed among a plurality of computational units wherein each unit processes a portion of the total computation.

Computer program instructions may be stored in a computer readable medium that can direct a computer, other programmable data processing apparatus, or other devices to function in a particular manner, such that the instructions stored in the computer readable medium produce an article of manufacture including instructions which implement the function/act specified in the disclosure.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically, a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASHEEPROM, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory or may be communicated to an external device so as to cause physical changes or actuation of the external device.

Broadly, embodiments of the present invention provide a sensor device for measuring bio-signals including one or more of electroencephalogram (EEG), electrocardiogram (ECG), heartbeat, electromyography (EMG), body temperature, body location, time, movement and velocity. The sensor device can include a circular fabric based headband with electrodes (conductive fabric sensing material) embedded therein, along with electronical circuitry and electrical wires. Electrodes can be positioned to make electrical contact with the scalp of a subject. For example, electroencephalography (EEG) measures the voltage changes resulting from ionic current with the neurons of the brain over a time period. By using multiple electrodes in contact with the scalp of the subject, neural oscillations can be detected in EEG measurements.

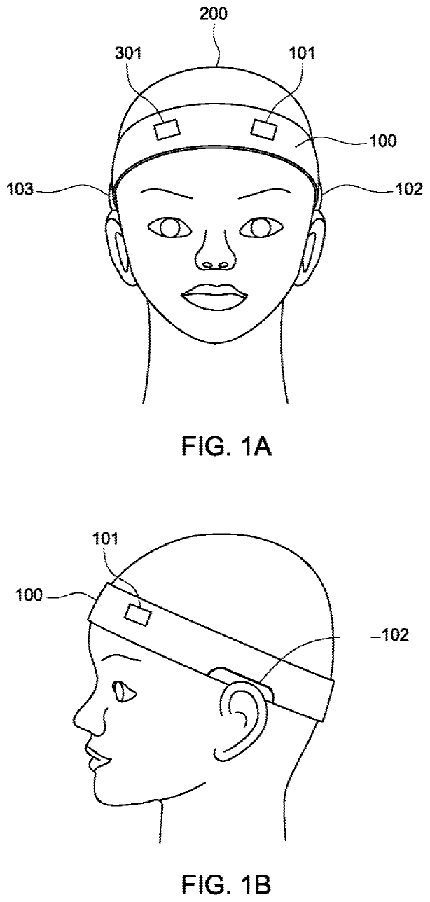

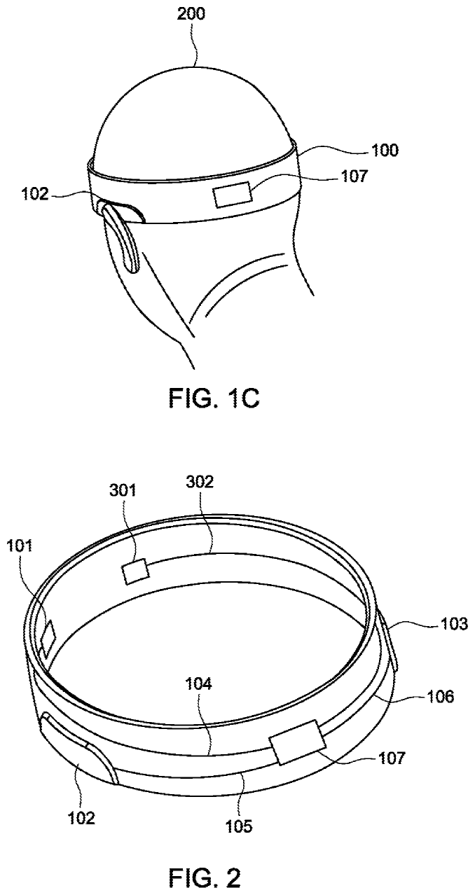

Referring to FIGS. 1A through 1C, various views of a brainwave headband 100 (also referred to as headband 100) are shown. In FIGS. 1A through 1C, the headband is shown on a user 200. One electrode contact 101 may be positioned, for example, on the left side of the user's forehead, which allows frontal lobe brain wave detection. Ground electrode contact 102 may be positioned at the user's left head side, and reference electrode contact 103 may be positioned at the user's right head side, both may be positioned to contact the user's ears. Electrode contacts may be made without the use of conducting gels or other liquids. Electrode contacts 101, 102 and 103 can include conductive fabrics to sense the electronic bio-signals. The back of the brainwave headband 100 can include an elastic fabric, allowing a single size band to accommodate most users. The front of the brainwave headband 100 may include an elastic or non-elastic fabric, allowing the active components to remain in a defined spacing with relation to each other. In some embodiments, the headband 100 can be formed from a two-layer construction, with an inner layer formed from an elastic material, and an outer layer, typically sandwiching the inner layer, but not attached thereto (or minimally attached thereto, such as fewer than four locations, for example). The outer layer may be bunched up when the elastic is not extended so that the elastic may be expanded while the outer layer may be non-elastic and/or non-stretching.

In some embodiments, the headband 100 may be formed of a fabric material that may be separated from the various electronic components, thus permitting the headband 100 to be washed.

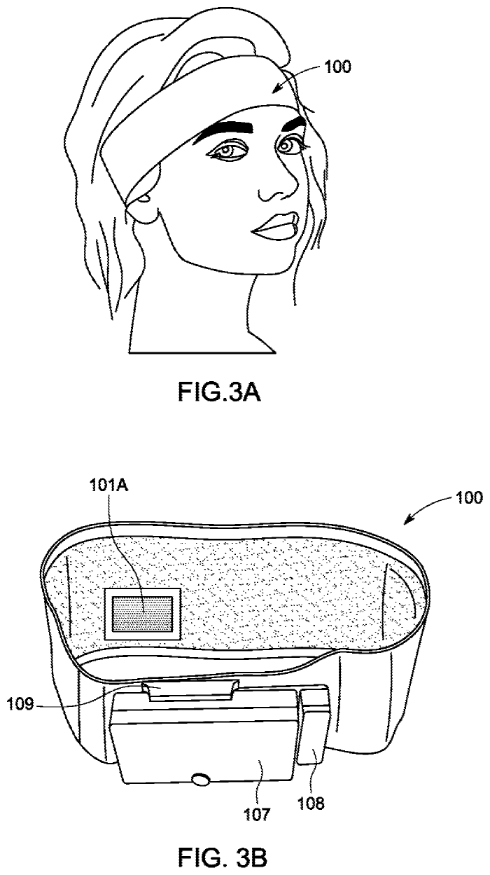

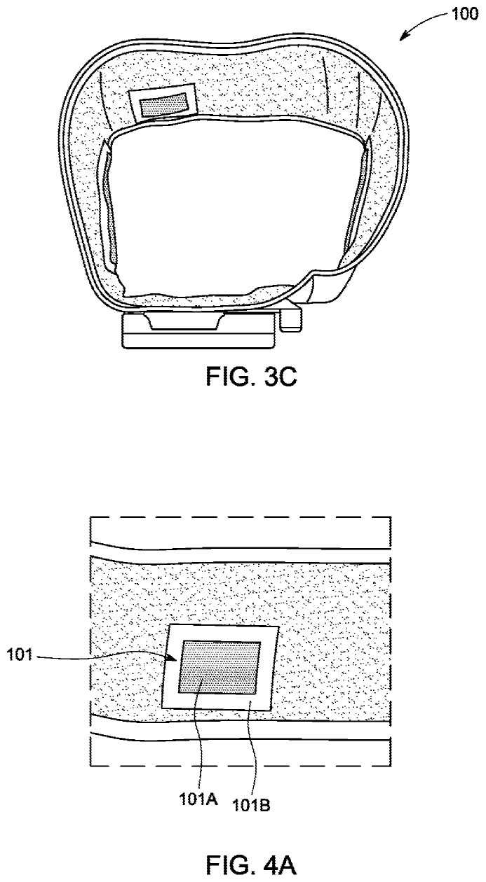

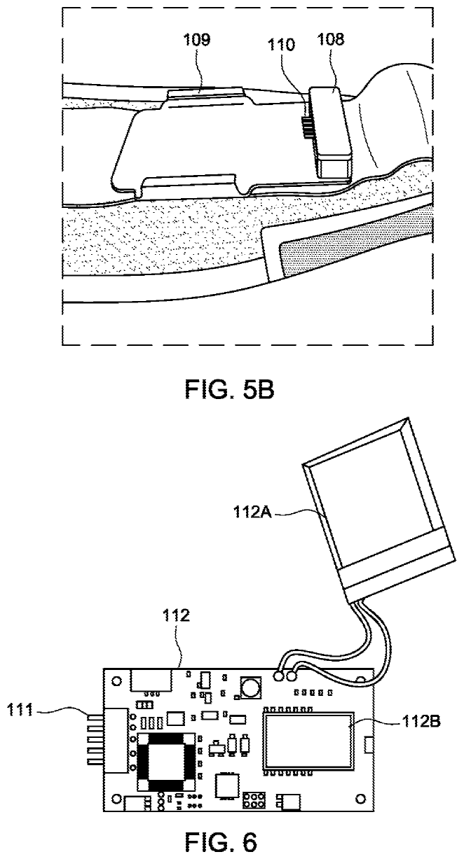

With reference to FIGS. 2 through 6, both inside and outside components are shown, with the connections among those components also displayed. With reference to FIGS. 2 and 3B, on the backside of the brainwave headband 100, there is a protective box 107 to hold a bio-signal detection PCBA module 112. The electrode contact 101 may be connected to protective box 107 and the inside to the PCBA module 112 through electronic shield wire 104. The electrode contact 102 may be connected to the protective box 107 and inside the PCBA module 112 through electronic shield wire 105. The electrode contact 103 may be connected to the protective box 107 and inside to the PCBA module 112 through electronic shield wire 106. With reference to FIGS. 3B, 3C, 5A and 5B, the protective box 107 may be docked with a plastic base 108 through lock schema 109. In some embodiments, the lock schema 109 can include side rails between which the protective box can slide 107. This particular embodiment may help align contacts 110 on the plastic base with a plug 111 on the PCBA module 112.

Depending on the different applications, for example, for a sleeping quality monitoring application, the protective box 107, with its internal components, and the relating plastic base 108, can be relocated in other areas of headband, such as at the front of the head, to allow users to have a good sleeping position. The headband internal wiring can be adjusted according to the desired relocation, but the electrode contact location can be located at the same location as described above. In some embodiments, a lead line (not shown) may connect to the contacts 110 on the plastic base and the protective box 107 may be remote therefrom, thus minimizing the size and the weight to be carried by the headband 100. In some embodiments, the protective box 107 (and its components, such as the PCBA module 112) may receive a wireless signal from the headband 100. In this embodiment, there is no need for a wired connection or a headband-mounted protective box 107.

As shown in FIG. 6, the PCBA module can include plug 111, circuitry operable to perform the various functions as described therein, a wireless communication module 112B, such as Bluetooth® module, and a power pack 112A, such as a rechargeable battery pack.

There may be a 5-signal male pogo pin connector 110 (also referred to above as contacts 110) and 5-signal female pogo pins 111 (also referred to above as plug 111) connected between the PCBA module 112 and the plastic base 108. The 5-signal male pogo pins 110 can be embedded in the plastic base 108. The 5-signal female pogo pins 111 can be disposed directly on the PCBA module 112. The pogo pins may be helpful to detect volts down to a level of about 10⁻⁶ which is typically not possible using a micro-USB port.

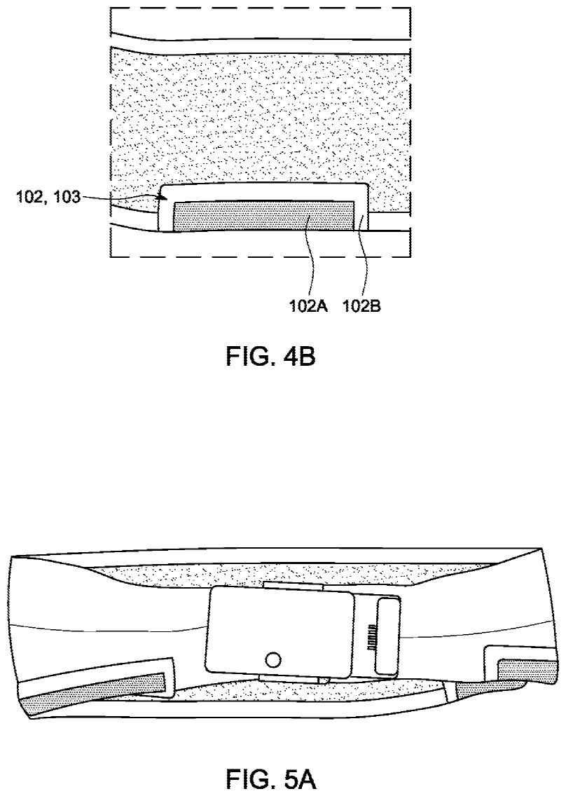

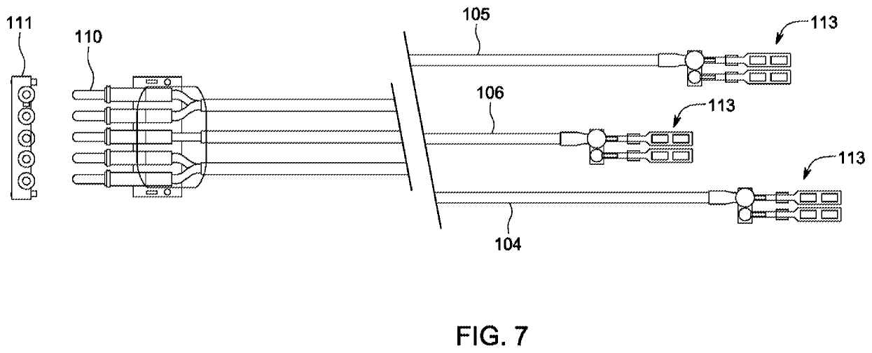

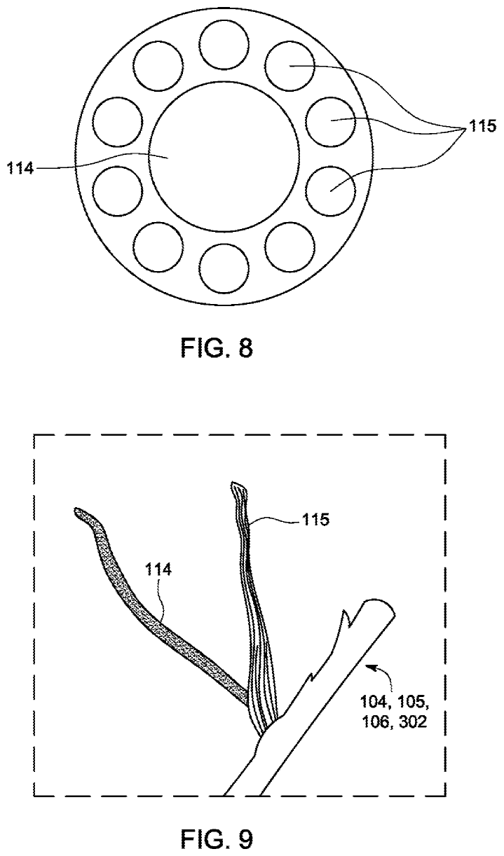

With reference to FIG. 7, 5-signal male pogo pins 110 can be soldered with 3 electronic shield wires 104, 105 and 106. With reference to FIGS. 8 and 9, center conductive wire 114 can be constructed with a diameter of 0.254 mm, specification as AWG30 (American wire gauge 30). Copper shield wires 115 can be constructed around center conductive wire 114 to provide superior noise immunity performance, which is desirable for EEG micro-voltage signal detection. The diameter of electronic shield wires 104, 105, 106 and 302 that include both center conductive wire 114 and copper shield wires 115 is 0.8 mm, for example. Silicone-sheathing wire is super-flexible and soft, able to handle up to 180° C. and up to 600V. This electronic wire can be specifically designed for wearable EEG product with high temperature endurance for volume production needs. In the manufacture, a high temperature pressure sticking process can be used to integrate headband 100 fabric materials with electrode contact 101, 102,103 conductive fabric sensors. As shown in FIG. 4A, in some embodiments, a perimeter 101B may be disposed about the electrically conducting fabric 101A of the contact 101. In this embodiment, if the headband 100 is formed from an elastic material, the region surrounding the contact 101 would minimally stretch due to the perimeter 101B being formed from a non-elastic, non-expanding material.

With reference to FIGS. 1A through 2, an auxiliary electrode contact 301 can provide additional sensing functionalities, including dual channel EEG detection, which is obtained from paring with the electrode contact 101. Plus, an embedded sensor can be included for temperature and heart rate detection. The auxiliary sensing data can be detected and sent to the PCBA module 112 through the electrode contact 302. If the user uses a finger of the left hand to touch the electrode contact 102, the electrocardiography (EEG) can be detected simultaneously with EEG signal. In some embodiments, auxiliary sensing data may be obtained via a secondary set of electrode, such as a heart rate and temperature set of electrodes, where the heart rate and temperature and ECG may be correlated, over time, to the EEG signal for determining the health and wellness of a user, for example.

With reference to FIG. 6, PCBA module 112 can include bio-sensing data process function, battery power charging and management function and wireless data transmitter (e.g. Bluetooth®) to allow the data to be transmitted and stored on a remote device. The PCBA module 112 can contain a memory (not specifically shown, but part of PCBA module 112) that records the bioelectric EEG signals and links this data to the corresponding measured ECG, body temperature and heart rate. In addition, a tri-axis gyroscope and accelerometer can be included on the PCBA module 112, which can be used for head movement detection. A Global Positioning Sensor (GPS) receiver can be disposed on the PCBA module 112 to allow location detection and time data collection. Thus, the location, time, movement and velocity of the head can all be recorded and correlated with brain signal and heart rate from a single device.

The headband bio-signal data could include (1) EEG measurement as detected by one or two electrodes and compared to a control/ground electrode; (2) time and location stamp of the EEG measurement; (3) correlated ECG, heart rate and body temperature; and (4) head motion/acceleration detection.

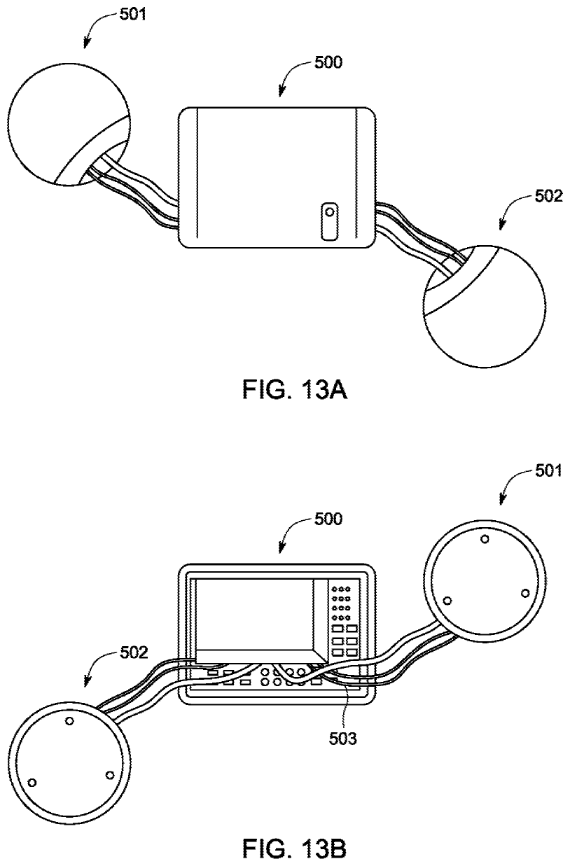

With reference to FIG. 13A and FIG. 13B, the free touch bio-signal detection module can include free touch electrode sensors 501 and 502, plus an electronic signal process unit box 500. Inside the box 500, there is signal process PCBA module 503, which can be integrated with PCBA module 112.

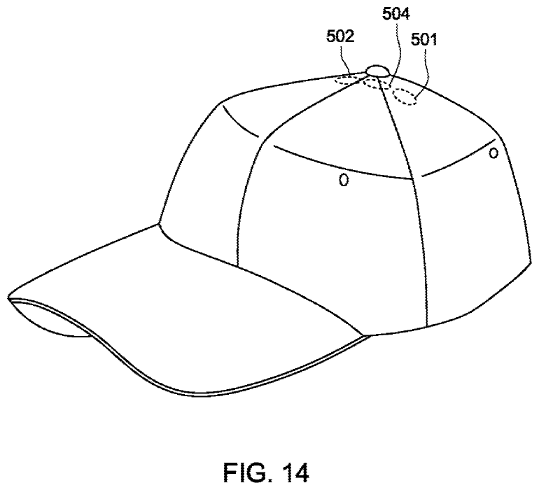

With reference to FIG. 14, the headband can be constructed inside a hat, and free touch bio-signal detection electrodes 504, 501 and 502 can be placed in a hairy area Cz (the center of the cerebral cortex), C3 (to the left of the cerebral cortex) and C4 (to the right of the cerebral cortex) which are considered to be optimal locations for recognizing motor imagery (MI) states.

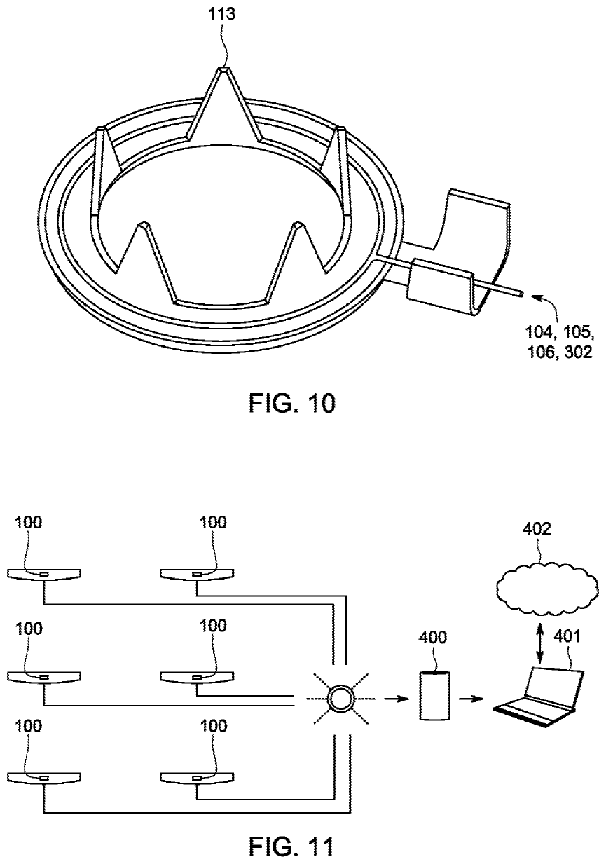

With reference to FIG. 10, a pin/conductive fabric solution 113 can be implemented to keep good signal quality and to provide ease of manufacture. The male pin pitch (top component) can be about 2.54 mm and the conductive fabric can be placed with pins pressuring down to keep a solid electrical signal connection. These pins can be used to bring the signal measured by electrode contacts 101, 102, 103, 301 in FIG. 2 to the PCBA module 112.

The present invention can provide for monitoring and analysis of bio-signal recordings for multiple brainwave headbands 100 worn by multiple persons at a specific location. As each headband 100 is equipped with embedded wireless transmission capability, such as Bluetooth®, Wi-Fi or other wireless communication module, the headband 100 can be used as a one-person system, with the definition as one user is using one headband 100 to get bio-signals and transfer those detection results through the wireless transmission module to an external device, such as a PC, laptop or mobile phone device.

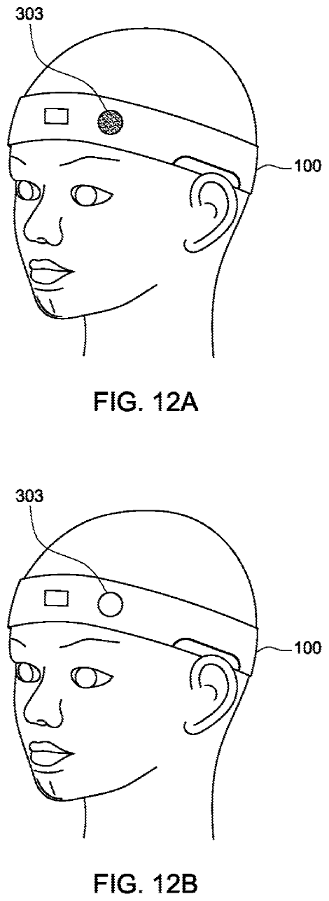

With reference to FIG. 11, since headband 100 is designed as a regular sports headband, multiple users can use the headbands 100 at the same time, which is defined as a multiple person bio-signal detection and analysis system. The simultaneous multiple persons' bio-signal data collection and data correlation among various bio-signals can provide valuable information about, for example, a user health status. The simultaneous multiple persons' bio-signal data can be collected through headband 100, and transferred through wireless communication, such as a Bluetooth® router 400, as shown in FIG. 11. The external processing device, such as a PC, mobile device or server 401, can process data and transfer the data to online clouding 402 for storage, further data analysis or transmission. In a third use mode, which may be defined as a “pairing mode”, with reference to FIGS. 12A and 12B, the headband 100 can communicate with each other directly. In this embodiment, a status indicator module 303 can provide a communication status indication in lighting, audio or video display format.

The multiple persons bio-signal data, as shown in FIG. 11, can be used by multiple users (e.g., students in a classroom) who wear the headbands 100 at the same time. In some embodiments, the system can track the brain functions of all students in real-time. This can help determine how efficiently the students are learning. The system could include a wireless centralized receiver, such as a Bluetooth® router, which could collect signals from, for example, 20 to 45 students concurrently. Readings could be sent to a server/cloud for further analyses. Each headband 100 could have to be specifically identified to avoid duplicates when pairing. This can be accomplished by using a MAC address, as discussed below, in each headband 100 which could be scanned and then paired.

One aspect of the present invention is a method for automatic pairing multiple brainwave headbands by using wireless MAC address and bio-signal signature feature. The signature feature is the particular bio-signal tag that belongs to the particular user, for example, the unique brainwave EEG bio-signal feature. There can be two stage pairing modes to be chosen. The first method is basic pairing mode that includes the pairing process with wireless module Media Access Control (MAC) address, which is a unique identifier (e.g. 0x88 0x1B 0x99 0x11 0x22 0x33) for a specific piece of the embedded wireless module. During the manufacture, each wireless module is programmed with MAC address to be broadcasted during wireless pairing mode. For example, one brainwave headband can be programmed with the following American Standard Code for Information Interchange (ASCII), 0x4D 0x41 0x88 0x1B 0x99 0x11 0x22 0x33, which stands for “MA881699112233” (Mindsall (MA) headband with Bluetooth® MAC address 881699112233).

When user uses the brainwave headband for the first time, the user needs to scan the barcode (the MAC address) label on the headband protective box 107, by using a mobile phone App designed for the brainwave headband. The mobile phone App can record the MAC address after scanning, and pair automatically with the headband Bluetooth® module. In this way, even if there are multiple headbands in the same location, external devices, such as a mobile phone, can still pair with one particular headband per user's desire. The second pairing mode involves the MAC address, and also the bio-signal detected signature feature results. After the first time wireless pairing, the App can record the user's unique bio-signal signature feature. For the next time pairing and operating, the App can check to see if user's bio-signal signature feature is presented in order for the communication to be continued.

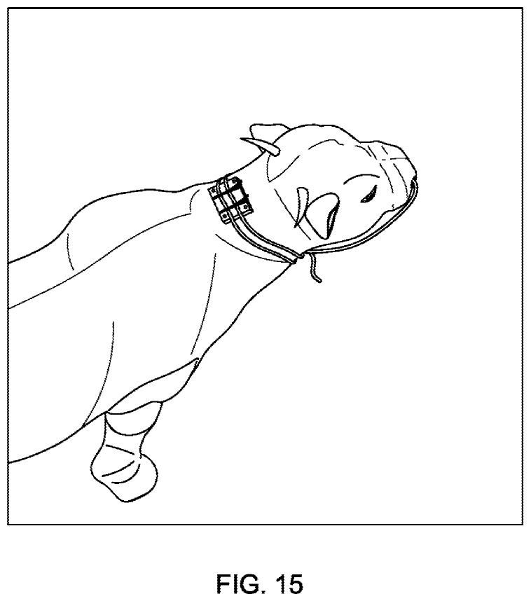

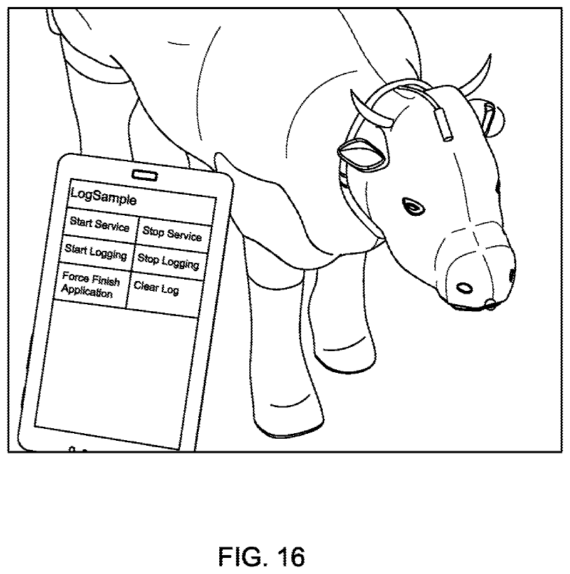

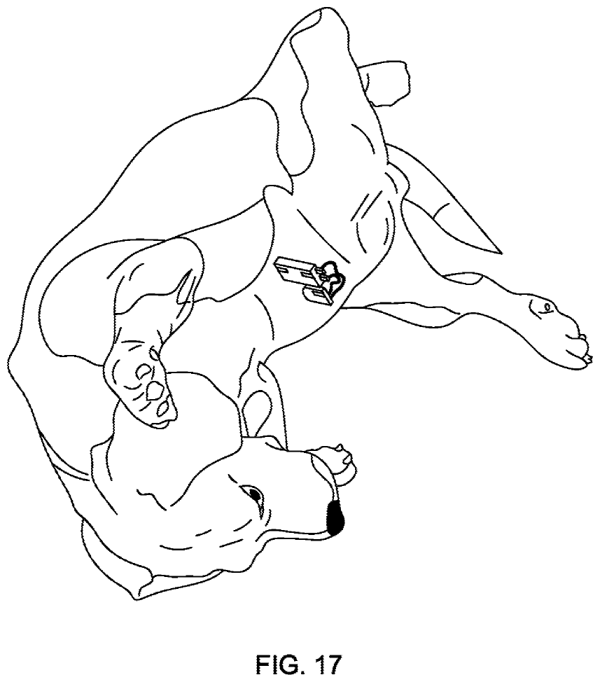

FIG. 15 shows a bio-signal detection PCB module that is attached on the cow's head. FIG. 16 shows sensors that can be pressured on the cow's head. The bio-signal data can be received through wireless communication and recorded by a tablet, for example.

Embodiments of the present invention may use the devices, as described above, on other animals or pets, such as a dog. A bio-signal device may be located in the area of the dog's lower abdomen as shown in the Figures.

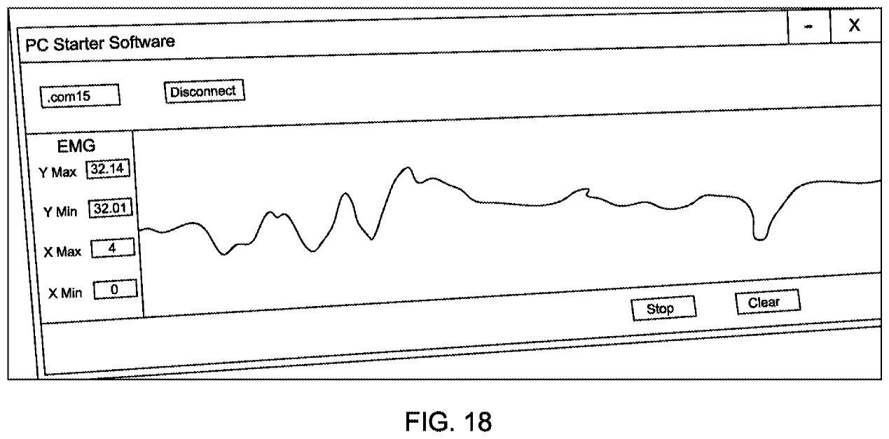

FIG. 18 illustrates testing results from a dog, which indicate that the sensor process has been feasible for animal bio-signal detection.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. A headband comprising;
a fabric band configured to fit about a user's head;
an electrode contact formed on an inside portion of the headband, the electrode contact configured to receive an electroencephalogram (EEG) signal from the user; and
a circuit for receiving the EEG signal from the electrode contact.

2. The headband of claim 1, wherein the electrode contact is formed from a conductive fabric.

3. The headband of claim 1, wherein the circuit is integrated into a protective box.

4. The headband of claim 3, wherein the protective box is removable from the fabric band via a connector.

5. The headband of claim 4, wherein the connector provides for the protective box to be removably attached to the headband.

6. The headband of claim 5, wherein the connector includes pogo pins.

7. The headband of claim 1, wherein the circuit is electrically connected to the electrode contact via a shielded wire having a central conductor surrounded by a plurality of shield wires.

8. The headband of claim 1, further comprising a wireless communication protocol for sending data from the circuit to an external computing device.

9. The headband of claim 1, further comprising memory for storing the EEG signal from the electrode contact.

10. The headband of claim 1, further comprising an auxiliary electrode connected to the circuit.

11. The headband of claim 10, wherein the auxiliary electrode provides a second EEG electrode for a dual channel EEG detection.

12. The headband of claim 11, wherein the auxiliary electrode provides a measurement of at least one of a user temperature and a heart rate detection.

13. The headband of claim 1, wherein the headband includes at least one side contact electrode formed along a bottom edge of the headband on at least one side thereof.

14. The headband of claim 1, wherein the circuit includes at least one of a gyroscope and an accelerometer for determining a user's head position and head movement.

15. A headband comprising;
a fabric band configured to fit about a user's head;
an electrode contact formed on an inside portion of the headband, the electrode contact configured to receive an electroencephalogram (EEG) signal from the user;
an auxiliary electrode contact formed on another inside portion of the headband, the auxiliary electrode contact configured to receive a second EEG signal from the user; and
a circuit for receiving the EEG signal from the electrode contact and the second EEG signal from the auxiliary electrode contact, the circuit correlating the EEG signal and the second EEG signal into a dual EEG.

16. The headband of claim 15, wherein the circuit is integrated into a protective box and the protective box is removable from the fabric band via a connector.

17. The headband of claim 16, wherein the connector includes pogo pins for removably connecting the protective box to the headband.

18. The headband of claim 15, further comprising:
memory for storing the EEG signal from the electrode contact and the second EEG signal from the auxiliary electrode contact; and
a wireless communication protocol for sending data from the circuit to an external computing device.

19. A bio-signal detecting device for measuring bio-signals including one or more of electroencephalogram (EEG), electrocardiogram (ECG), heartbeat, electromyography (EMG), body temperature, body location, time, movement and velocity, comprising:
a wearable element configured to be worn by a user;
an electrode contact formed on an inside portion of the headband, the electrode contact configured to receive an electroencephalogram (EEG) signal from the user;
one or more embedded sensors, embedded in at least one of the electrode contact and
a circuit for receiving the EEG signal from the electrode contact.

20. The bio-signal detecting device of claim 19, wherein the wearable element is selected from the group consisting of a headband and a hat.

21. The bio-signal detecting device of claim 19, wherein the one or more embedded sensors is a free touch sensor.

22. The bio-signal detecting device of claim 19, wherein the user is one of a human and an animal.

23. The bio-signal detecting device of claim 19, further comprising:
an electrode contact formed on an inside portion of the wearable element, the electrode contact configured to receive an electroencephalogram (EEG) signal from the user;
an auxiliary electrode contact formed on another inside portion of the wearable element, the auxiliary electrode contact configured to receive a second electronic signal from the user; and
the circuit is configured for receiving the EEG signal from the electrode contact, the second signal from the auxiliary electrode contact and a signal from the one or more embedded sensors.
